(19) United States
(12) Patent Application Publication
(10) Pub. No.: US 2018/0325456 A1
(43) Pub. Date: Nov. 15, 2018

(54) MULTI-TIME SCALE WAVEFORM FOR DISPLAY OF SENSOR MEASUREMENTS

(71) Applicant: Boston Scientific Scimed Inc., Maple Grove, MN (US)

(72) Inventors: Daniel Klebanov, Arlington, MA (US); Nicholas Herlambang, Waltham, MA (US); Ruslan R. Hristov, Lexington, MA (US); Jacob I. Laughner, St. Paul, MN (US); Mordechai Perlman, Cambridge, MA (US)

(21) Appl. No.: 15/975,352

(22) Filed: May 9, 2018

Related U.S. Application Data
(60) Provisional application No. 62/504,230, filed on May 10, 2017.

(51) Int. Cl. A61B 5/00; A61B 18/14; A61B 5/042; A61B 5/053

(52) U.S. Cl. CPC A61B 5/6885; A61B 18/1492; A61B 2090/065; A61B 5/0538; A61B 5/6852; A61B 5/0422

(57) ABSTRACT

Medical devices and method for generating multi-time scale waveforms for display of sensor measurements are disclosed. Sensed or measured output signals from a sensor, such as a catheter, are processed to generate a first data set that uses a first time scale and a second data set that uses a second time scale. The generated data sets are then displayed on a display by juxtaposing the first data set with the second data set. In this manner, measurement data from the sensor can be shown in dual time scales that allow for faster and more efficient visual diagnostic assessments.

### CROSS-REFERENCE TO RELATED APPLICATION

This application claims priority to Provisional Application No. 62/504,230, filed May 10, 2017, which is herein incorporated by reference in its entirety.

### TECHNICAL FIELD

The present invention relates to medical devices and methods for tissue diagnosis and/or ablation. More specifically, the present invention relates to devices and methods for monitoring tissue contact between a sensor and tissue within the body.

### BACKGROUND

Cardiac arrhythmia and/or other cardiac pathology contributing to abnormal heart function may originate in cardiac cellular tissue. One technique utilized to treat cardiac arrhythmia and/or cardiac pathology is ablation of the tissue substrates contributing to the arrhythmia and/or cardiac pathology. The tissue substrates can be electrically disrupted, or ablated, by heat, chemicals or other means of creating a lesion in the tissue, or otherwise can be electrically isolated from the normal heart circuit. Electrophysiology therapy involves locating the tissue contributing to the arrhythmia and/or cardiac pathology using a sensor, such as a catheter, and then using the sensor (or another device) to destroy and/or isolate the tissue.

Prior to performing an ablation procedure, physicians and/or clinicians may utilize specialized mapping and/or diagnostic sensors or catheters to precisely locate the tissue contributing and/or causing the arrhythmia or other cardiac pathology. It may, therefore, be desirable to be able to precisely locate the targeted tissue prior to performing the ablation procedure in order to effectively alleviate and/or eliminate the arrhythmia and/or cardiac pathology. Further, precise targeting of the tissue can prevent or reduce the likelihood that healthy tissue (located proximate the targeted tissue) is damaged.

Several techniques have been employed to precisely locate the targeted tissue where an ablation or other therapeutic procedure is performed. One example utilizes an ablation, mapping and/or diagnostic catheter to determine how close the catheter is to the targeted tissue. Further, the ablation, mapping and/or diagnostic catheter can include one or more sensing electrodes located on a distal portion of the catheter. The electrodes can sense, measure and/or provide a controller with information relating to the electrical activity within the cardiac tissue. Using the sensed and/or measured electrical information, the controller is able to correlate the spatial location of the distal portion of the catheter in relation to the cardiac tissue. For example, the electrodes can measure the impedance, resistance, voltage potential, etc. and determine how far the distal portion of the catheter is to the cardiac tissue.

The sensed and/or measured electrical information from the catheter can be displayed using a graph to provide visual diagnostic assessments. Conventional graphs usually depict any sensed and/or measured electrical information (e.g., impedance) over a single time scale. However, there is an ongoing need to provide enhanced graphs that can display additional information to allow for faster and more efficient visual diagnostic assessments.

### SUMMARY

In Example 1, a medical system comprising a processor and a display unit. The processor is configured to receive a first signal from a sensor positioned on or in proximity to an anatomical feature of a patient, and to process the first signal to generate a second signal representing a first parameter to be displayed. The display unit is configured to display the first parameter as a first continuous waveform within a single display window. The single display window has a first display region configured to display the continuous waveform using a first time scale, and a second display region configured to display the first continuous waveform using a second time scale that is different than the first time scale.

In Example 2, the medical system of Example 1, wherein the first region of the single display window is configured to represent a more recent time period than the second region of the display period.

In Example 3, the medical system of any of Examples 1-2, wherein the single display window includes a transition between the first region and the second window, and further wherein a position of the transition within the single display window is selectably adjustable by a user of the medical system.

In Example 4. the medical system of any of Examples 1-3, wherein the first time scale is defined at least in part by a first time interval, and the second time scale is defined at least in part by a second time interval, and wherein the first time interval is shorter than the second time interval.

In Example 5, the medical system of any of Examples 1-4, wherein the processor is further configured to process the first signal to generate a third signal representing a second parameter to be displayed, and wherein the display unit is further configured to display the second parameter as a second continuous waveform within the single display window concurrently with the first continuous waveform.

In Example 6, the medical system of Example 5, wherein the third signal is a rolling average of the first signal.

In Example 7, the medical system of Example 6, wherein the processor is configured to generate the third signal by applying a filter to one or both of the first signal and the second signal.

In Example 8, the medical system of any of Examples 1-7, wherein the sensor is an electrode and the anatomical feature is a myocardial tissue within a chamber of the patient's heart, and wherein the first signal is a voltage and the first parameter is an electrical impedance of the myocardial tissue.

In Example 9, the medical system of Example 8, further comprising a catheter, wherein the sensor is disposed on the catheter, and wherein the electrical impedance of the myocardial tissue is indicative of a degree of contact between a distal portion of the catheter and the myocardial tissue.

In Example 10, the medical system of any of either of Examples 8 or 9, wherein the sensor is a sensing electrode, and wherein the catheter further includes a current-injecting electrode for applying a current to the myocardial tissue, and wherein the first signal is a voltage sensed by the sensing electrode in response to the current applied to the myocardial tissue by the current-injecting electrode.

In Example 11, the medical system of either of Examples 9 or 10, wherein the catheter is a radiofrequency (RF) ablation catheter, and wherein the distal portion of the catheter includes an RF ablation electrode.

In Example 12, the medical system of Example 11, wherein the RF ablation electrode is the current-injecting electrode.

In Example 13, the medical system of either of Examples 11 or 12, wherein the sensing electrode is disposed within and electrically isolated from the RF ablation electrode.

In Example 14, the medical system of Examples 9 or 10, wherein the catheter is a mapping catheter, and wherein the distal portion of the mapping catheter includes the sensor.

In Example 15, the medical system of any of Examples 1-6, wherein the sensor is a temperature sensor and the anatomical feature is a myocardial tissue within a chamber of the patient's heart, and wherein the first parameter is a temperature of the myocardial tissue.

In Example 16, a method comprising receiving, by a processor, a first signal from a sensor positioned on or in proximity to an organ of a patient, and processing, by the processor, the first signal to generate a second signal representing a first parameter. The method further comprises outputting the second signal to a display unit, and displaying the first parameter as a continuous waveform within a single display window of the display unit. The single display window includes a first display region that displays the continuous waveform using a first time scale, and a second display region that displays the continuous waveform using a second time scale that is different than the first time scale.

In Example 17, the method of Example 16, wherein the first region of the single display window represents a more recent time period than the second region of the display period.

In Example 18, the method of Example 16, wherein the single display window includes a transition between the first region and the second window, and the method further comprises a user adjusting a position of the transition within the single display window.

In Example 19, the method of Example 16, wherein the first time scale is defined at least in part by a first time interval, and the second time scale is defined at least in part by a second time interval, and wherein the first time interval is shorter than the second time interval.

In Example 20, the method of Example 16, further comprising processing the first signal to generate a third signal representing a second parameter to be displayed, and displaying the second parameter as a second continuous waveform within the single display window concurrently with the first continuous waveform.

In Example 21, the method of Example 20, wherein the third signal is a rolling average of the first signal.

In Example 22, the method of Example 20, wherein processing the first signal to generate the third signal includes applying a filter to one or both of the first signal and the second signal.

In Example 23, the method of Example 16, wherein the sensor is an electrode and the anatomical feature is a myocardial tissue within a chamber of the patient's heart, and wherein the first signal is a voltage and the first parameter is an electrical impedance of the myocardial tissue.

In Example 24, the method of Example 23, wherein the sensor is disposed on a catheter, and wherein the electrical impedance of the myocardial tissue is indicative of a degree of contact between a distal portion of the catheter and the myocardial tissue.

In Example 25, the method of Example 24, wherein the sensor is a sensing electrode, and wherein the catheter further includes a current-injecting electrode for applying a current to the myocardial tissue, and wherein the first signal is a voltage sensed by the sensing electrode in response to the current applied to the myocardial tissue by the current-injecting electrode.

In Example 26, the method of Example 25, wherein the catheter is a radiofrequency (RF) ablation catheter, and wherein the distal portion of the catheter includes an RF ablation electrode, and wherein the RF ablation electrode is the current-injecting electrode.

In Example 27, a medical system comprising a processor and a display unit. The processor is configured to receive a first signal from a sensor positioned on or in proximity to an anatomical feature of a patient, and to process the first signal to generate a second signal representing a first parameter to be displayed. The display unit is configured to display the first parameter as a first continuous waveform within a single display window. The single display window has a first display region configured to display the continuous waveform using a first time scale defined at least in part by a first time interval, and a second display region configured to display the first continuous waveform using a second time scale defined at least in part by a second time interval. The first time interval is shorter than the second time interval.

In Example 28, the medical system of Example 27, wherein the single display window includes a transition between the first region and the second window, and further wherein a position of the transition within the single display window is selectably adjustable by a user of the medical system.

In Example 29, the medical system of Example 27, wherein the processor is further configured to process the first signal to generate a third signal representing a second parameter to be displayed, and wherein the display unit is further configured to display the second parameter as a second continuous waveform within the single display window concurrently with the first continuous waveform.

In Example 30, the medical system of Example 27, wherein the sensor is an electrode and the anatomical feature is a myocardial tissue within a chamber of the patient's heart, and wherein the first signal is a voltage and the first parameter is an electrical impedance of the myocardial tissue.

In Example 31, the medical system of Example 30, further comprising a radiofrequency (RF) ablation catheter, wherein the sensor is disposed on the RF ablation catheter, and wherein the electrical impedance of the myocardial tissue is indicative of a degree of contact between a distal portion of the RF ablation catheter and the myocardial tissue.

In Example 32, an apparatus for displaying a multi-time scale waveform. The apparatus comprises a processor configured to receive output signals from a sensor, process the output signals to generate a first data set using a first time scale, process the output signals to generate a second data set using a second time scale, and provide for display of the generated data sets in a single display window on a display unit by juxtaposing the first data set using the first time scale with the second data set using the second time scale.

In Example 33, the apparatus of Example 32, wherein time intervals of the first time scale are smaller than time intervals of the second time scale.

In Example 34, the apparatus of Example 33, wherein the first data set using the first time scale flows into the second data set using the second time scale from right to left at a transition point that is configurable by a user.

In Example 35, the apparatus of Example 32, wherein the processor is further configured to provide for display a third data set using the first time scale by averaging the first data set using the first time scale, and provide for display a fourth data set using the second time scale by averaging the second data set using the second time scale.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a block diagram of an example cardiac mapping and/or ablation system including an ablation catheter according to one embodiment.

FIG. 2 is an example graphical representation of a display window for use in the medical apparatus of FIG. 1 according to one embodiment.

FIGS. 3 and 4 are graphical representations of alternative displays within a single display window for use in the medical apparatus of FIG. 1 according to embodiments.

FIG. 5 is a flow diagram of an example method for generating a display of an output signal from a sensor on the ablation catheter of FIG. 1 according to an embodiment.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

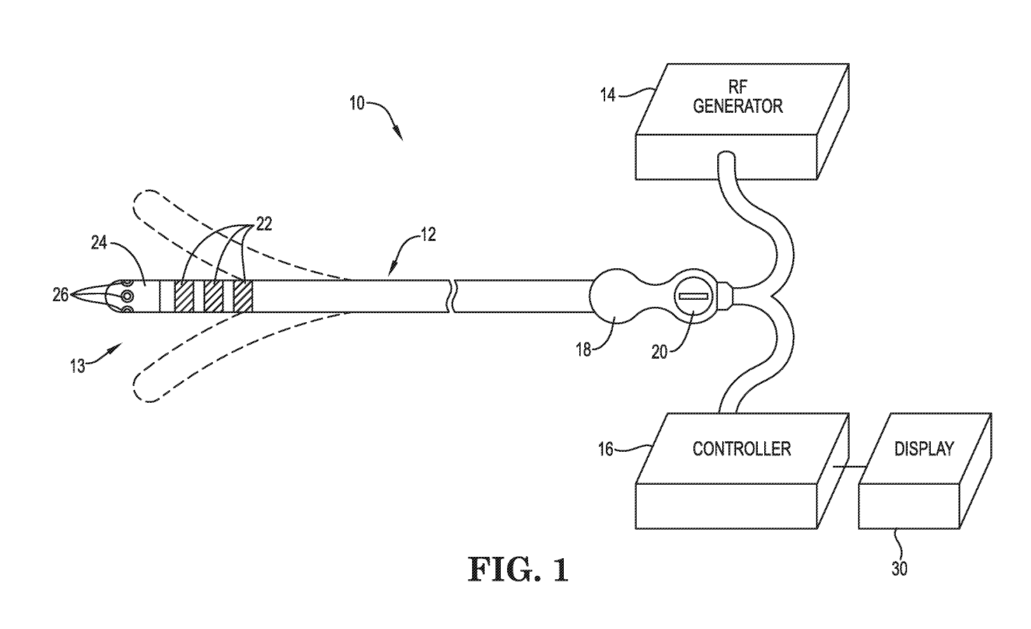

FIG. 1 illustrates an example medical system 10 which, in the illustrated embodiment, is a cardiac mapping and/or ablation system. As shown in FIG. 1, the system 10 includes an elongated member or catheter shaft 12, an RF generator 14, a controller 16 (e.g., a mapping processor, ablation processor, and/or other processor), and a display unit 30. In one embodiment, the system 10 can be any of the devices and systems described in U.S. patent application Ser. No. 14/881,112, entitled “TISSUE DIAGNOSIS AND TREATMENT USING MINI-ELECTRODES” and filed Oct. 12, 2015, the contents of which are incorporated by reference herein.

However, the system 10 is not limited to any of the foregoing devices and systems, and may be configured in accordance with any cardiac mapping and/or ablation system, whether now known or later developed, having the capabilities and functionality described therein.

Alternatively, in some embodiments, the various embodiments may be comprised of medical systems other than cardiac mapping and/or ablation systems. In general, as will be discussed in greater detail elsewhere herein, embodiments of the invention may be used with any medical system in which a display of physiological information, as derived from one or more sensors, in a single window using two or more time scales, can provide useful clinical information to the clinician.

In the embodiment of FIG. 1, the shaft 12 is operatively coupled to at least one or more (e.g., one or both) of the RF generator 14 and the controller 16. Alternatively, or in addition, a device (other than shaft 12), may be utilized to apply ablation energy and/or diagnose a target area and may be operatively coupled to at least one or more of the RF generator 14 and the controller 16. The RF generator 14 is configured for delivering ablation energy to the shaft 12 in a controlled manner in order to ablate target area sites within a subject's heart. In the various embodiments, the controller 16 includes a processor, a memory and/or any other components as known in the art.

Although the controller 16 and the RF generator 14 are shown as discrete components, these components or features of components can be incorporated into a single device.

In some embodiments, the shaft 12 includes a handle 18, which has an actuator 20 (e.g., a control knob or other actuator). The handle 18 is positioned at a proximal end of the shaft 12, for example. The shaft 12 includes a flexible body having a distal portion 13 which includes one or more electrodes. For example, in the illustrated embodiment, the distal portion 13 of the shaft 12 includes one or more of a plurality of ring electrodes 22, a distal ablation tip electrode 24, and a plurality of mini-electrodes 26 disposed or otherwise positioned along and/or electrically isolated from the distal ablation tip electrode 24. In the various embodiments, any of the ring electrodes 22, the distal ablation tip electrode 24 and the mini-electrodes 26 can be operable, at least in part, as sensors to sense electrical signals associated with cardiac tissue to which they are in proximity or in contact when in use.

The shaft 12 can be steered to facilitate navigating the vasculature of a patient or navigating other lumens. The distal portion 13 of the shaft 12 can be deflected by manipulation of the actuator 20 to affect steering of the shaft 12. In some instances, the distal portion 13 of the shaft 12 is deflected to position the distal ablation tip electrode 24 and/or the mini-electrodes 26 adjacent a target tissue or to position the distal portion 13 of the shaft 12 for another suitable purpose. Additionally or alternatively, the distal portion 13 of the shaft 12 can have a pre-formed shape adapted to facilitate positioning the distal ablation tip electrode 24 and/or the mini-electrodes 26 adjacent a target tissue. The pre-formed shape of the distal portion 13 of the shaft 12 can be a radiused shape (e.g., a generally circular shape or a generally semi-circular shape) and/or can be oriented in a plane transverse to a general longitudinal direction of the shaft 12.

In some embodiments, the system 10 is utilized in ablation procedures on a patient. The shaft 12 is configured to be introduced into or through the vasculature of a patient and/or into or through any other lumen or cavity. In one example, the shaft 12 is inserted through the vasculature of the patient and into one or more chambers of the patient's heart (e.g., a target area). When in the patient's vasculature or heart, the shaft 12 is used to map and/or ablate myocardial tissue using the ring electrodes 22, the mini-electrodes 26, and/or the distal ablation tip electrode 24. In some instances, the distal ablation tip electrode 24 is configured to apply ablation energy to the myocardial tissue of the heart of a patient.

As stated, the mini-electrodes 26 can be circumferentially distributed about the distal ablation tip electrode 24. The mini-electrodes 26 are capable of operating, or configured to operate, in unipolar or bipolar sensing modes. The mini-electrodes 26 are capable of sensing, or can be configured to sense, electrical characteristics (e.g., impedance) corresponding to the myocardial tissue proximate thereto.

In the various embodiments, the system 10 is capable of provide impedance tissue measurements to allow the user to assess contact between the catheter tip (e.g., the distal ablation tip electrode 24) and tissue. In general, the impedance of a given medium is measured by applying a known voltage or current to a given medium and measuring the resulting voltage or current. In other words, impedance measurements of a given medium can be obtained by injecting current between two electrodes and measuring the resulting voltage between the same electrodes through which the current was injected. The ratio of the voltage potential to the applied current provides an indication of the impedance of the medium through which the current traveled.

For example, in one embodiment, a current can be injected between the distal ablation tip electrode 24 and the ring electrodes 22. Impedance of the medium (e.g., tissue) adjacent to the distal ablation tip electrode 24 and the ring electrodes 22 can be measured according to the methodology disclosed above. For example, if the distal ablation tip electrode 24 and the ring electrodes 22 are embedded in cardiac tissue, the impedance of the cardiac tissue can be determined.

In some instances, the system 10 utilizes different impedance measurements of a local medium to determine whether the distal ablation tip electrode 24 is contacting tissue. For example, the impedance of cardiac tissue is different than that of blood. Therefore, by knowing the relative difference in the impedance of tissue versus blood, the system 10 can determine whether the medium through which a current is being applied is either blood or cardiac tissue, for example.

In some examples, the mini-electrodes 26 are operatively coupled to the controller 16. Further, the generated output from the mini-electrodes 26 is sent to the controller 16 for processing. As stated, an electrical characteristic (e.g., impedance) and/or an output signal from a mini-electrode pair can at least partially form the basis of a contact assessment, ablation area assessment (e.g., tissue viability assessment), and/or an ablation progress assessment (e.g., a lesion formation/maturation analysis).

Further, the system 10 is capable of processing or can be configured to process the electrical signals from the mini-electrodes 26, the ring electrodes 22, and/or the distal ablation tip electrode 24. Based, at least in part, on the processed output from the mini-electrodes 26, the ring electrodes 22, and/or the distal ablation tip electrode 24, the controller 16 generates an output to the display unit 30 (e.g., a monitor, a screen, or any other image projection device) for visual diagnostic assessments by a physician or other user. In instances where an output is generated to the display unit 30, the controller 16 is operatively coupled to or otherwise in communication with the display unit 30. The display unit 30 can include various static and/or dynamic information related to the use of the system 10. In one example, the display unit 30 includes one or more of an image of the target area, an image of the shaft 12, and/or indicators conveying information corresponding to tissue proximity, which can be analyzed by a user and/or by a processor of the system 10 to determine the existence and/or location of arrhythmia substrates within the heart, to determine the location of the shaft 12 within the heart, and/or to make other determinations relating to use of the shaft 12 and/or other elongated members.

In various embodiments, the system 10 is configured such that the display unit 30 can provide to the user, in a single display window, a graphical representation of a parameter of interest, e.g., tissue impedance indicative of electrode/tissue contact, as a continuous waveform using two or more different time scales, each defined by a different time interval. Displaying the selected parameter in this manner can, among other things, assist the user in ascertaining the degree and/or stability of the electrode/tissue contact, and in discerning whether impedance changes are resulting from other causes (e.g., normal cardiac wall motion, respiratory cycle effects, or changes in tissue impedance caused by the formation of lesions during the ablation procedure).

In some embodiments, the system 10 may include an indicator in communication with the controller 16. The indicator may be capable of providing an indication related to a feature of the output signals received from one or more of the electrodes 22, 24, 26 of the shaft 12. In one example, an indication to a physician about a characteristic of the shaft 12 and/or the myocardial tissue interacted with and/or being mapped may be provided on the display 30. In some cases, the indicator may provide a visual and/or audible indication to provide information concerning the characteristic of the shaft 12 and/or the myocardial tissue interacted with and/or being mapped. For example, the system 10 can determine that a measured impedance corresponds to an impedance value of cardiac tissue and therefore outputs a color indicator (e.g., green) to the display 30. The color indicator may allow a physician to more easily determine whether to apply ablative therapy to a given cardiac location.

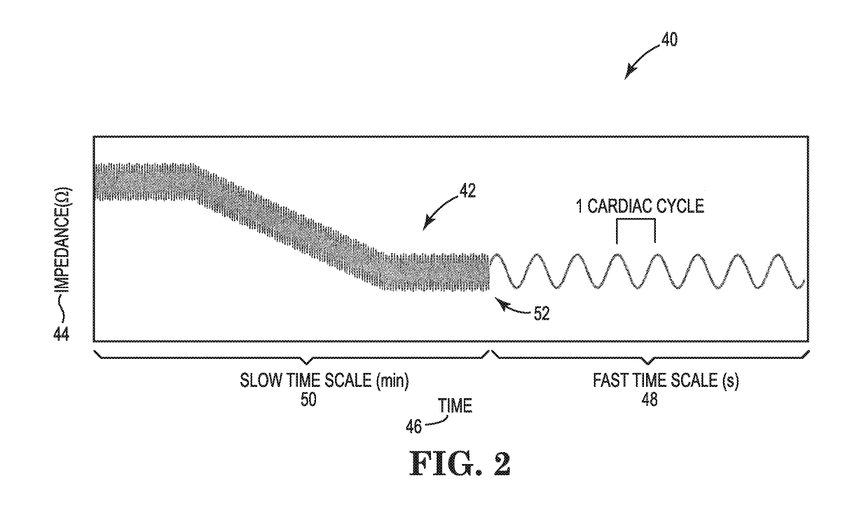

FIG. 2 illustrates an example graphical representation 40 of a display window 40 of the display unit 30 according to one embodiment. In the illustrated embodiment the display window 40 displays the parameter of interest (in this case, myocardial tissue impedance) in the form of a continuous, multi-time scale waveform 42. As stated, electrical signals from one or more of the electrodes 22, 24, 26 of the shaft 12 in FIG. 1 can be processed for providing a signal to the display unit 30, and consequently the display window 40, representing the electrical impedance of the myocardial tissue proximate the catheter shaft 12. In one example, the controller 16 processes the electrical signals from the mini-electrodes 26, the ring electrodes 22, and/or the distal ablation tip electrode 24 in the shaft 12 to generate an output signal to be displayed in the single display window 40 as the multi-time scale or multi-speed waveform 42 as shown in FIG. 2.

The waveform 42 is in the form of a sweep graph that juxtaposes two different time scales. As shown in FIG. 2, a vertical axis 44 depicts the amplitude of the sensed and/or measured data, while a horizontal axis 46 depicts time. In one example, the vertical axis 44 may represent measured tissue impedance (in Ohms). Accordingly, the waveform 42 allows a user (e.g., a physician or clinician) to quickly visualize, in real-time, changes in tissue impedance in dual time scales.

For example, FIG. 2 illustrates two useful time scales on the horizontal axis 46, each corresponding to a different region within the display window 40. The first time scale is a fast time scale 48, which shows immediate changes in tissue impedance. This is used to indicate the stability of sensor or catheter positioning. The fast time scale 48 may be visualized over several cardiac cycles. A typical cardiac cycle is on the order of half a second. As such, a useful range for the fast time scale 48 can be approximately 5 to 10 seconds.

The second time scale is a slow time scale 50, which shows changes in tissue impedance over extended periods of time. As such, a useful range for the slow time scale 50 can be anywhere from 2 to 10 minutes. Note that a drop in the tissue impedance in the slow time scale 50 represents the occurrence of an ablation, which may last from 30 seconds to several minutes.

In the illustrated embodiment, the waveform 42 is represented by a continuous stream of data. Thus, as new data is received, the entire waveform 42 scrolls or sweeps from right to left. In other words, data in the fast time scale 48 moves leftward into the slow time scale 50 at a transition point 52. The transition point 52 is adjustable by the user. Moreover, both the fast time scale and the slow time scale are adjustable by the user.

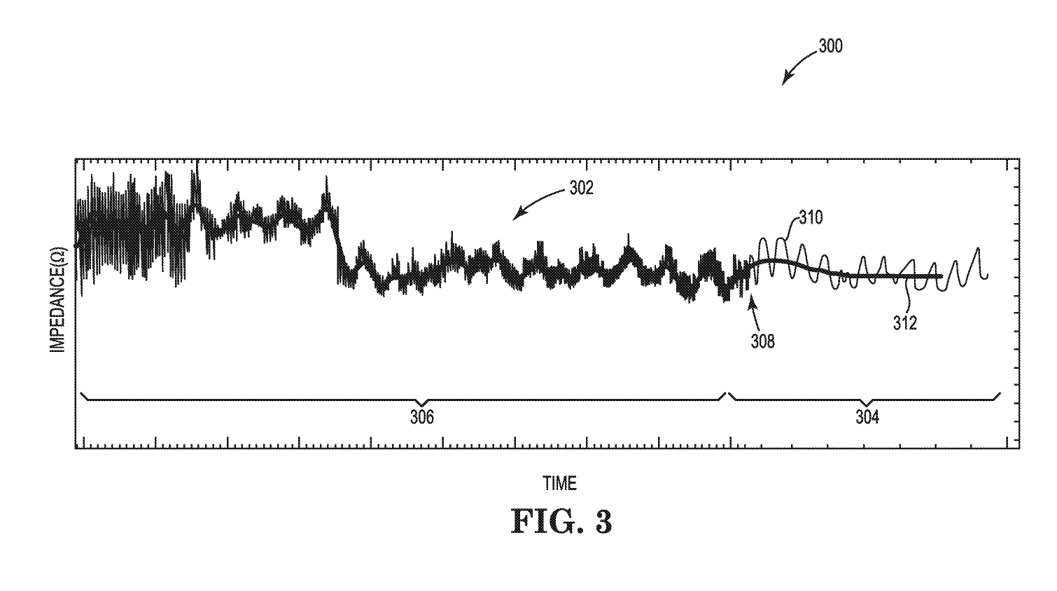

FIG. 3 illustrates an exemplary display window 300 that can be included in the display unit 30 of the system 10 according to another embodiment. As illustrated, shown in the display window 300 is a a multi-time scale waveform 302 is visualized as measured tissue impedance (in Ohms) over time. In the illustrated embodiment, the waveform 302 is indicative of the electrical impedance of myocardial tissue that has not been ablated (i.e., there is no ablation lesion present in the tissue proximate the sensing electrode(s)). As shown, the waveform 302 has a first data region 304 with data shown in a fast time scale (in seconds), and a second data region 306 with data shown in a slow time scale (in minutes). A transition point 308 indicates the flow of data from the first data region 304 to the second data region 306 (or from the fast time scale into the slow time scale). FIG. 3 also shows a trace 310 representing actual data and a trace 312 representing an averaged version of the actual data. In some cases, a low-pass or another type of filter can be used to obtain the trace 312.

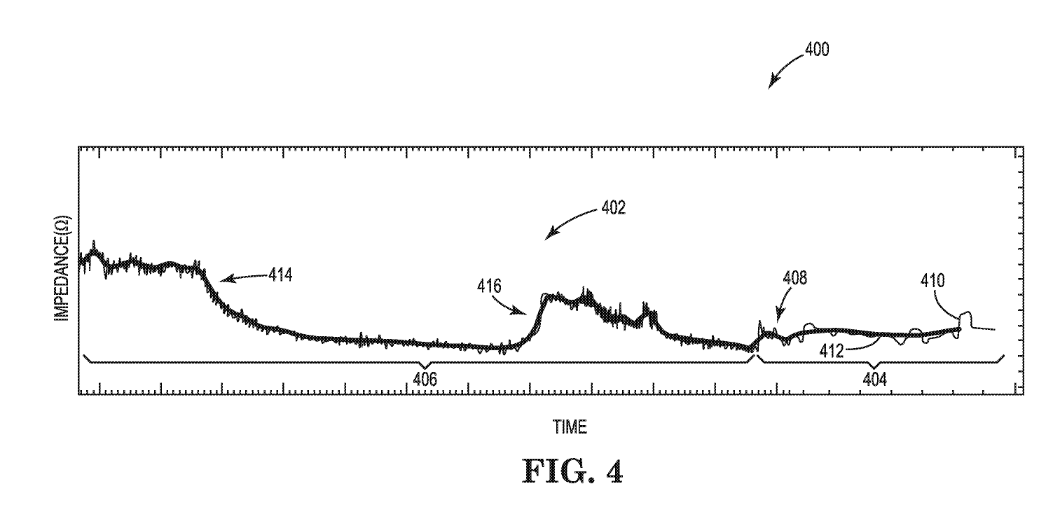

FIG. 4 illustrates an exemplary display window that can be included in the display unit 30 of the system 10 according to another embodiment. As shown in FIG. 4, a multi-time scale waveform 402 is visualized as measured tissue impedance (in Ohms) over time. The waveform 402 has a first data region 404 with data shown in a fast time scale (in seconds), and a second data region 406 with data shown in a slow time scale (in minutes). A transition point 408 indicates the flow of data from the first data region 404 to the second data region 406 (or from the fast time scale into the slow time scale). FIG. 4 also shows a trace 410 representing actual data and a trace 412 representing an averaged version of the actual data. Further, the formation of an ablation lesion in the affected tissue can be ascertained from the waveform 402. Specifically, as can be seen in FIG. 4, in the second data region 406, a drop 414 and a subsequent rise 416 in the measured tissue impedance indicates the formation of an ablation lesion at the location of the sensing electrode(s).

As can be seen in FIGS. 3 and 4, the first data regions 304, 404, respectively, correspond to relatively fast time scales defined by relatively short time intervals (as indicated by the spacing of the tick marks on the horizontal axis in those regions). In contrast, in the second data regions 306, 406, the corresponding time scales are defined by relatively longer time intervals. As will be appreciated, the specific time intervals can be selected based on the particular parameter being displayed. In addition, in the various embodiments, the respective time intervals may be user-selectable, as can be the particular location of the transition points 308, 408 within the respective display windows 300, 400,

Furthermore, although in FIGS. 2, 3 and 4, the display windows 40, 300 and 400 each include two display regions with corresponding time scales, in other embodiments, more than two display regions/time scales can be employed.

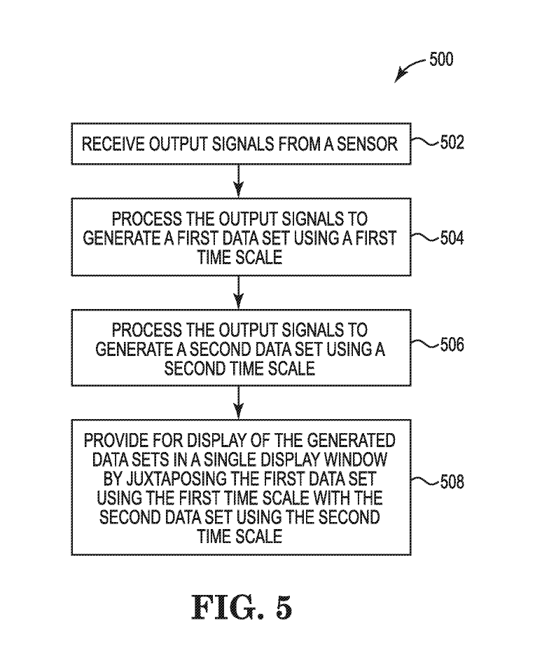

FIG. 5 is a flow diagram that illustrates an example method 500 for generating a multi-time scale waveform. The method 500 may be performed by a controller and/or a processor in a controller (e.g., the controller 16 of FIG. 1).

The method 500 includes receiving output signals from a sensor (block 502). The sensor may be, for example, a catheter (e.g., the catheter 12 of FIG. 1) or other types of sensor. As such, the output signals received from the sensor can include impedance measurements, resistance measurements, voltage measurements, current measurements, force measurements, temperature measurements (e.g., temperature readings from thermistors or thermocouples on a catheter), and/or electrogram measurements (e.g., amplitudes and reduction over time). In some embodiments, the output signals can include indications of ablation power and/or on/off indications for an ablation catheter. The output signals can also include indications for catheter slip detection. Further, the output signals can include computed values that correlate to lesion size formation, such as a force-time integral or a future metric based on impedance measurements.

The method 500 also includes processing the received output signals to generate a first data set using a first time scale (block 504) and processing the received output signals to generate a second data set using a second time scale (block 506). The first time scale is different from the second time scale. Specifically, the time intervals of the first time scale are less or smaller than the time intervals of the second time scale. For example, the first time scale may have time intervals based on seconds whereas the second time scale may have time intervals based on minutes. In some cases, the first time scale and the second time scale can be selected or are configurable by a user.

The method 500 also includes providing for display of the generated data sets in a single display window by juxtaposing the first data set using the first time scale with the second date set using the second time scale (block 508). For example, the method 500 may send the generated data sets to be displayed on a display (e.g., the display 30 of FIG. 1). In some instances, the generated data sets may be displayed via a user interface on the display.

In displaying the generated data sets, the first data set using the first time scale can be displayed in a first region on the display, while the second data set using the second time scale can be displayed in a second region on the display. The first data set using the first time scale can flow or transition into the second data set using the second time scale from right to left at a transition point (see FIG. 2). The transition point can be selected or is configurable by a user.

Moreover, the method 500 can display a third data set that uses the first time scale by averaging the first data set using the first time scale. The method 500 can also display a fourth data set that uses the second time scale by averaging the second data set using the second time scale.

It should be noted that, for simplicity and ease of understanding, the elements described above and shown in the figures are not drawn to scale and may omit certain features. As such, the drawings do not necessarily indicate the relative sizes of the elements or the non-existence of other features.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A method comprising:
receiving, by a processor, a first signal from a sensor positioned on or in proximity to an organ of a patient;
processing, by the processor, the first signal to generate a second signal representing a first parameter;
outputting the second signal to a display unit;
displaying the first parameter as a continuous waveform within a single display window of the display unit, wherein the single display window includes a first display region that displays the continuous waveform using a first time scale, and a second display region that displays the continuous waveform using a second time scale that is different than the first time scale.

2. The method of claim 1, wherein the first region of the single display window represents a more recent time period than the second region of the display period.

3. The method of claim 1, wherein the single display window includes a transition between the first region and the second window, and the method further comprises a user adjusting a position of the transition within the single display window.

4. The method of claim 1, wherein the first time scale is defined at least in part by a first time interval, and the second time scale is defined at least in part by a second time interval, and wherein the first time interval is shorter than the second time interval.

5. The method of claim 1, further comprising processing the first signal to generate a third signal representing a second parameter to be displayed, and displaying the second parameter as a second continuous waveform within the single display window concurrently with the first continuous waveform.

6. The method of claim 5, wherein the third signal is a rolling average of the first signal.

7. The method of claim 5, wherein processing the first signal to generate the third signal includes applying a filter to one or both of the first signal and the second signal.

8. The method of claim 1, wherein the sensor is an electrode and the anatomical feature is a myocardial tissue within a chamber of the patient's heart, and wherein the first signal is a voltage and the first parameter is an electrical impedance of the myocardial tissue.

9. The method of claim 8, wherein the sensor is disposed on a catheter, and wherein the electrical impedance of the myocardial tissue is indicative of a degree of contact between a distal portion of the catheter and the myocardial tissue.

10. The method of claim 9, wherein the sensor is a sensing electrode, and wherein the catheter further includes a current-injecting electrode for applying a current to the myocardial tissue, and wherein the first signal is a voltage sensed by the sensing electrode in response to the current applied to the myocardial tissue by the current-injecting electrode.

11. The method of claim 10, wherein the catheter is a radiofrequency (RF) ablation catheter, and wherein the distal portion of the catheter includes an RF ablation electrode, and wherein the RF ablation electrode is the current-injecting electrode.

12. A medical system comprising:
a processor configured to receive a first signal from a sensor positioned on or in proximity to an anatomical feature of a patient, and to process the first signal to generate a second signal representing a first parameter to be displayed; and
a display unit configured to display the first parameter as a first continuous waveform within a single display window, wherein the single display window has a first display region configured to display the continuous waveform using a first time scale defined at least in part by a first time interval, and a second display region configured to display the first continuous waveform using a second time scale defined at least in part by a second time interval, and wherein the first time interval is shorter than the second time interval.

13. The medical system of claim 12, wherein the single display window includes a transition between the first region and the second window, and further wherein a position of the transition within the single display window is selectably adjustable by a user of the medical system.

14. The medical system of claim 12, wherein the processor is further configured to process the first signal to generate a third signal representing a second parameter to be displayed, and wherein the display unit is further configured to display the second parameter as a second continuous waveform within the single display window concurrently with the first continuous waveform.

15. The medical system of claim 12, wherein the sensor is an electrode and the anatomical feature is a myocardial tissue within a chamber of the patient's heart, and wherein the first signal is a voltage and the first parameter is an electrical impedance of the myocardial tissue.

16. The medical system of claim 15, further comprising a radiofrequency (RF) ablation catheter, wherein the sensor is disposed on the RF ablation catheter, and wherein the electrical impedance of the myocardial tissue is indicative of a degree of contact between a distal portion of the RF ablation catheter and the myocardial tissue.

17. An apparatus for displaying a multi-time scale waveform comprising:
a processor configured to:
receive output signals from a sensor;
process the output signals to generate a first data set using a first time scale;
process the output signals to generate a second data set using a second time scale; and
provide for display of the generated data sets in a single display window on a display unit by juxtaposing the first data set using the first time scale with the second data set using the second time scale.

18. The apparatus of claim 17, wherein time intervals of the first time scale are smaller than time intervals of the second time scale.

19. The apparatus of claim 18, wherein the first data set using the first time scale flows into the second data set using the second time scale from right to left at a transition point that is configurable by a user.

20. The apparatus of claim 17, wherein the processor is further configured to provide for display a third data set using the first time scale by averaging the first data set using the first time scale, and provide for display a fourth data set using the second time scale by averaging the second data set using the second time scale.
